(19) United States
(12) Patent Application Publication
(10) Pub. No.: US 2015/0162770 A1
(43) Pub. Date: Jun. 11, 2015

(54) APPARATUS AND METHOD FOR MANAGING HEARING DEVICE

(71) Applicant: SAMSUNG ELECTRONICS CO., LTD., Suwon-si (KR)

(72) Inventors: Jong Min CHOI, Seoul (KR); Hee Suk SHIN, Daejeon (KR); Ji Yeon JUNG, Suwon-si (KR)

(73) Assignee: SAMSUNG ELECTRONICS CO., LTD., Suwon-si (KR)

(21) Appl. No.: 14/561,685

(22) Filed: Dec. 5, 2014

(30) Foreign Application Priority Data
Dec. 9, 2013 (KR) 10-2013-0152592

(51) Int. Cl. H02J 7/00; B08B 5/02; A61L 2/18; B08B 5/04; H04R 25/00; A61L 2/24; F26B 21/06; B08B 1/04

(52) U.S. Cl. CPC H02J 7/0052; F26B 21/06; B08B 5/02; B08B 1/04; B08B 5/04; H04R 25/50; A61L 2/24; A61L 2/18; H02J 7/0047

(57) ABSTRACT

An apparatus for managing a hearing device through interoperation with a control terminal is provided. The apparatus may control, through an external control terminal, constituent elements configured to perform various operations of drying, cleaning, sterilizing, charging, and setting the hearing device.

### CROSS-REFERENCE TO RELATED APPLICATION

This application claims the benefit under 35 USC 119(a) of Korean Patent Application No. 10-2013-0152592, filed on Dec. 9, 2013, in the Korean Intellectual Property Office, the entire disclosure of which is incorporated herein by reference for all purposes.

### BACKGROUND

1. Field

The following description relates to a station for storing a hearing device, and a method of controlling various operations of the station through a control terminal, which is disposed outside the station.

2. Description of Related Art

A hearing device such as, for example, a hearing aid may be stored in a separate space when it is not in use after being worn in an ear of a user. Similar to a case for glasses, devices currently used to store hearing aids are configured to simply protect the hearing aid with an enclosure. In addition to storing a hearing aid, various operations to maintain the functions of the hearing aid may be needed. With development of network devices, there is also a demand for a method to confirm a state of a hearing aid to manage the hearing aid.

### SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

In one general aspect, there is provided an apparatus for managing a hearing device including a drying unit configured to dry the hearing device disposed in the apparatus, and a communication unit configured to receive a control instruction from a control terminal to adjust an operating parameter of the drying unit.

The apparatus may include a display configured to indicate an operating state of the drying unit is disposed on the station.

The operating parameter of the drying unit may include at least one of a drying time, a drying strength, a drying period, a drying timer setting, or a drying state of the hearing device.

The apparatus may include a lighting device configured to display different colors based on an operating state of the drying unit.

In another general aspect, there is provided an apparatus for managing a hearing device including a cleaning unit configured to clean the hearing device disposed in the apparatus, and a communication unit configured to receive a control instruction from a control terminal to adjust an operating parameter of the cleaning unit.

The operating parameter of the cleaning unit may include at least one of an airflow strength, a suction strength, a rotation velocity of a brush to clean the hearing device, a cleaning state, a cleaning period, or a cleaning timer setting of the hearing device.

The apparatus may include a display configured to indicate an operating state of the cleaning unit.

The apparatus may include a lighting device configured to display different colors based on an operating state of the cleaning unit.

In another general aspect, there is provided an apparatus for managing a hearing device including a sterilizing unit configured to sterilize the hearing device disposed in the apparatus, and a communication unit configured to receive a control instruction from a control terminal to adjust an operating parameter of the sterilizing unit.

The operating parameter of the sterilizing unit includes at least one of a sterilizing time, a sterilizing period, a disinfectant replacement, a sterilizing timer setting, or a sterilizing state of the hearing device.

The apparatus may include a display configured to indicate an operating state of the sterilizing unit.

The apparatus may include a lighting device configured to display different colors based on an operating state of the sterilizing unit.

In another general aspect, there is provided an apparatus for managing a hearing device including a charging unit configured to charge a hearing device disposed in the apparatus, and a communication unit configured to receive a control instruction from a control terminal to adjust an operating parameter of the charging unit.

The operating parameter of the charging unit may include at least one of a charging time, a charging completion, a charging period, a charging timer setting, and a charging state of the hearing device.

The apparatus may include a display configured to indicate an operating state of the charging unit.

The apparatus may include a lighting device configured to display different colors based on an operating state of the charging unit.

In another general aspect, there is provided an apparatus for managing a hearing device including a setting unit configured to set an application of a hearing device disposed in the apparatus, and a communication unit configured to receive a control instruction from a control terminal to adjust an operating parameter of the setting unit.

The operating parameter of the setting unit may include at least one of a setting of a parameter or a firmware upgrade of the hearing device.

The apparatus may include a display configured to indicate an operating state of the setting unit.

The apparatus may include a lighting device configured to display different colors based on an operating state of the setting unit.

Other features and aspects will be apparent from the following detailed description, the drawings, and the claims.

### BRIEF DESCRIPTION OF THE DRAWINGS

FIG. 1 is a diagram illustrating an example of a station for a hearing device.

FIG. 2 is a diagram illustrating an example of a station.

FIG. 3 is a diagram illustrating an example of a configuration of a station.

FIG. 4 is a diagram illustrating an example of functions of a control terminal to dry a hearing device.

FIG. 5 is a diagram illustrating an example of functions of a control terminal to clean a hearing device.

FIG. 6 is a diagram illustrating an example of functions of a control terminal to sterilize a hearing device.

FIG. 7 is a diagram illustrating an example of functions of a control terminal to charge a hearing device.

FIG. 8 is a diagram illustrating an example of functions of a control terminal to set a hearing device.

Throughout the drawings and the detailed description, unless otherwise described or provided, the same drawing reference numerals will be understood to refer to the same elements, features, and structures. The drawings may not be to scale, and the relative size, proportions, and depiction of elements in the drawings may be exaggerated for clarity, illustration, and convenience.

### DETAILED DESCRIPTION

The following detailed description is provided to assist the reader in gaining a comprehensive understanding of the methods, apparatuses, and/or systems described herein. However, various changes, modifications, and equivalents of the systems, apparatuses, and/or methods described herein will be apparent to one of ordinary skill in the art. The progression of processing steps and/or operations described is an example; however, the sequence of and/or operations is not limited to that set forth herein and may be changed as is known in the art, with the exception of steps and/or operations necessarily occurring in a certain order. Also, descriptions of functions and constructions that are well known to one of ordinary skill in the art may be omitted for increased clarity and conciseness.

The features described herein may be embodied in different forms, and are not to be construed as being limited to the examples described herein. Rather, the examples described herein have been provided so that this disclosure will be thorough and complete, and will convey the full scope of the disclosure to one of ordinary skill in the art.

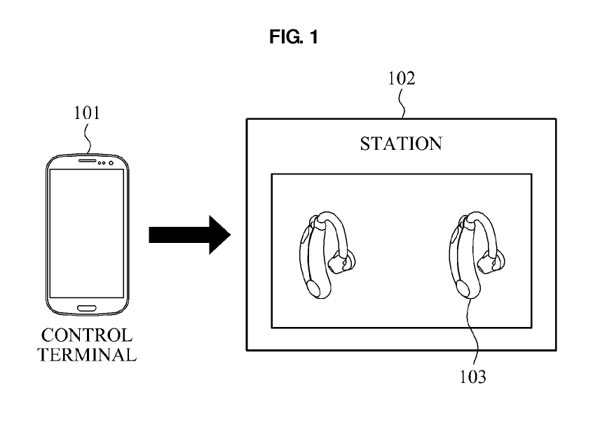

FIG. 1 is a diagram illustrating an example of a station 102 for a hearing device 103. Referring to FIG. 1, the hearing device 103 is stored in the station 102. The station 102 may store the hearing device 103, and perform other operations. For example, station 102 may dry, clean charge, sterilize, and set the hearing device 103. The station 102 may be provided in a fixed form, or in a small-sized form, which is portable.

The various operations of the station 102 may be controlled based on a control instruction that is transferred from a control terminal 101. The control terminal 101 may be disposed outside the station 102, and may transfer the control instruction to the station 102 via a network. As a non-exhaustive illustration only, the control terminal 101 described herein may refer to mobile devices such as, for example, a cellular phone, a smart phone, a wearable smart device (such as, for example, a ring, a watch, a pair of glasses, glasses-type device, a bracelet, an ankle bracket, a belt, a necklace, an earring, a headband, a helmet, a device embedded in the cloths or the like), a personal computer (PC), a tablet personal computer (tablet), a phablet, a mobile internet device (MID), a personal digital assistant (PDA), an enterprise digital assistant (EDA), a digital camera, a digital video camera, a portable game console, an MP3 player, a portable/personal multimedia player (PMP), a handheld e-book, an ultra mobile personal computer (UMPC), a portable lab-top PC, a global positioning system (GPS) navigation, a personal navigation device or portable navigation device (PND), a handheld game console, an e-book, and devices such as a high definition television (HDTV), an optical disc player, a DVD player, a Blue-ray player, a setup box, robot cleaners, or any other device capable of wireless communication or network communication consistent with that disclosed herein. The control terminal 101 may transfer the control instruction to the station 102 using various types of communication methods, such as, for example, Ethernet, near field communication (NFC), wireless-fidelity (Wi-Fi), Bluetooth, and radio frequency identification (RFID).

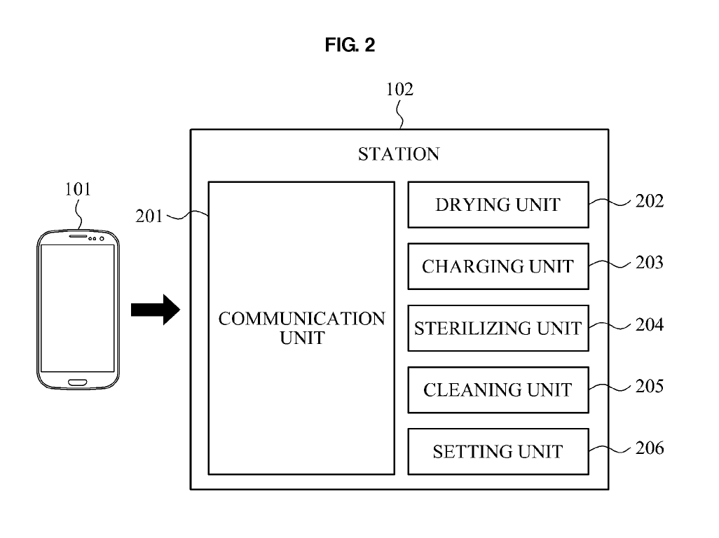

FIG. 2 is a diagram illustrating an example of the station 102. Referring to FIG. 2, the station 102 includes a communication unit 201. The station 102 may further include a drying unit 202, a charging unit 203, a sterilizing unit 204, a cleaning unit 205, and a setting unit 206.

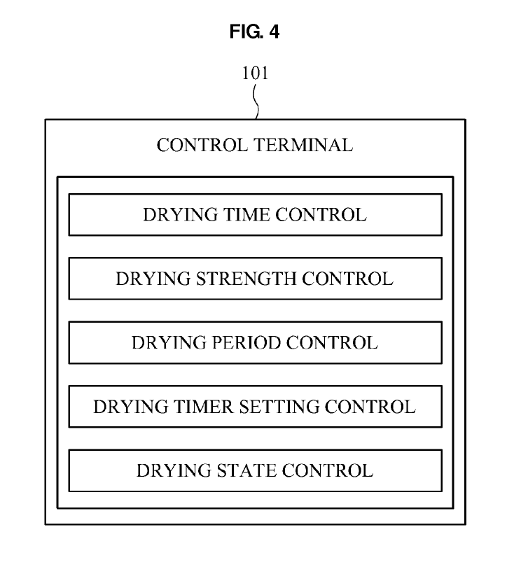

The communication unit 201 may receive a control instruction from the control terminal 101 via a network to adjust an operating parameter of the drying unit 202, the charging unit 203, the sterilizing unit 204, the cleaning unit 205, or the setting unit 206. Operating parameters of the drying unit 202, the cleaning unit 205, the sterilizing unit 204, the charging unit 203, and the setting unit 206 will be described with reference to FIGS. 4 through 8.

The drying unit 202 may dry a hearing device disposed in a station 102. The charging unit 203 may charge a hearing device disposed in a station 102. The sterilizing unit 204 may sterilize a hearing device disposed in a station 102. The cleaning unit 205 may clean a hearing device disposed in a station 102. The setting unit 206 may set an application of a hearing device disposed in a station 102. In an example, the cleaning unit 203 may be disposed in the sterilizing unit 204 or the sterilizing unit 204 may be adapted to perform the functions of the cleaning unit 203.

The station 102 may provide various functions related to a hearing device, and the functions may be controlled through the control terminal 101, which may be disposed outside the station 102.

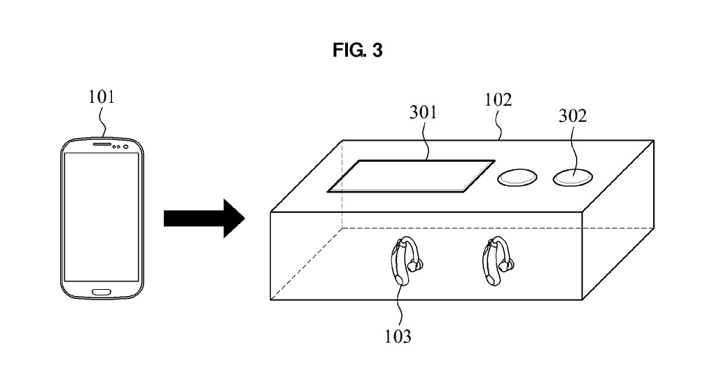

FIG. 3 is a diagram illustrating an example of a configuration of the station 102. Referring to FIG. 3, an information window 301 may indicate an operating state of the drying unit 202, the charging unit 203, the sterilizing unit 204, the cleaning unit 205, or the setting unit 206. The operating state may include detailed setting information of the operations of the station 102, a status of the progress of one or more of the operations, and a message indicating the completion of the operation related to the drying unit 202, the charging unit 203, the sterilizing unit 204, the cleaning unit 205, or the setting unit 206.

Various operating states of the drying unit 202, the charging unit 203, the sterilizing unit 204, the cleaning unit 205, or the setting unit 206 to be used to manage the hearing device 103 disposed in the station 102 may be displayed through the information window 301. The information window 301 may be implemented as a liquid crystal display (LCD), a light-emitting diode (LED) display, a thin film transistor (TFT-LCD) display, a light emitting diode (LED) display, an organic LED (OLED) display, an active matrix OLED (AMOLED) display, a flexible display, a plasma display panel (PDP), a screen, a terminal, and the like. A screen may be a physical structure that includes one or more hardware components that provide the ability to render a user interface and/or receive user input. The screen can encompass any combination of display region, gesture capture region, a touch sensitive display, and/or a configurable area. The screen can be embedded in the station 102 or may be an external peripheral device that may be attached and detached from the station 102. The display may be a single-screen or a multi-screen display. A single physical screen can include multiple displays that are managed as separate logical displays permitting different content to be displayed on separate displays although part of the same physical screen.

A lighting device 302, which is configured to express different colors based on the operating state of the drying unit 202, the charging unit 203, the sterilizing unit 204, the cleaning unit 205, or the setting unit 206 may be installed on the station 102. For example, the lighting device 302 may emit red light when one of the drying unit 202, the charging unit 203, the sterilizing unit 204, the cleaning unit 205, and the setting unit 206 is in operation, and may emit green light when the operation is terminated. Although not shown in FIG. 3, the station 102 may be configured to change a cover design thereof through a simple switch control.

FIG. 4 is a diagram illustrating an example of the functions of the control terminal 101 to dry a hearing device. Referring to FIG. 4, the control terminal 101 may control various functions of the drying unit 202. Such functions may be displayed through a display provided in the control terminal 101, and a user may control the functions of the drying unit 202 through an input of the control terminal 101.

The control terminal 101 may control a drying time, a drying strength, a drying period, a drying timer setting, and a drying state of the hearing device. A hearing device is vulnerable to moisture and thus, drying the hearing device is beneficial. A humidity sensor, which is provided in the station 102 may measure a humidity of the hearing device and provide the measured humidity to the control terminal 102 via a network. The humidity sensor may measure the humidity of the hearing device it may be difficult to verify, with naked eyes, whether moisture has been adequately removed through drying.

The drying time refers to an amount of time during which a hearing device is to be dried with air of preset temperature. The drying strength refers to a speed or temperature of air to be used to dry a hearing device. The drying period refers to a period that is set to dry a hearing device with air of preset temperature. The drying timer setting refers to a timer setting for the drying unit 202 to initiate the operation of drying a hearing device with air of preset temperature at a predetermined point in time. The drying state indicates whether the drying of a hearing device is completed, and a level of a drying state of the hearing device.

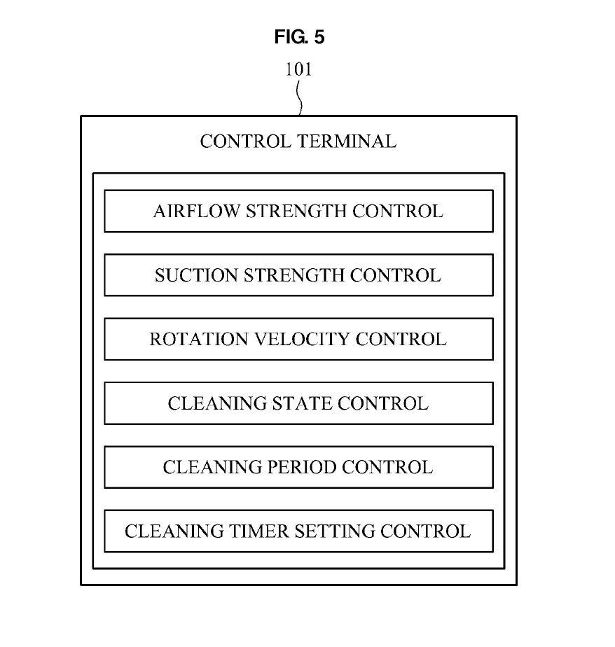

FIG. 5 is a diagram illustrating an example of the functions of the control terminal 101 to clean a hearing device. Referring to FIG. 5, the control terminal 101 may control various functions of the cleaning unit 205. Such functions may be displayed through a display provided in the control terminal 101, and a user may control the functions of the cleaning unit 205 through an input of the control terminal 101.

The control terminal 101 may control an airflow strength, a suction strength, a rotation velocity, a cleaning state, a cleaning period, and a cleaning timer setting of the hearing device. The hearing device is worn in an ear of a human and thus, there may be earwax on the hearing device. The earwax is unsanitary and may cause malfunction of the hearing device. Thus, the hearing device is cleaned to remove the earwax.

The airflow strength refers to a speed of air to be blown toward a hearing device to remove the earwax. The suction strength refers to a speed of suction air to be applied to a hearing device to remove the earwax. The rotation velocity refers to a rotation velocity of a brush that is in contact with a hearing device to remove the earwax. The cleaning state may refer to an amount of earwax removed from a hearing device, a progress of a cleaning operation of the cleaning unit 205, or whether the cleaning operation of the cleaning unit 205 is completed. The cleaning period control refers to a period that is set to clean a hearing device. The cleaning timer setting refers to a timer setting for the cleaning unit 205 to initiate cleaning a hearing device at a predetermined point in time.

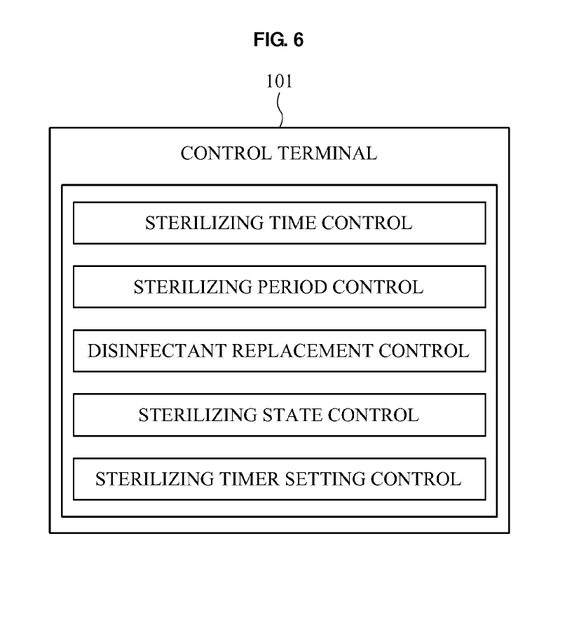

FIG. 6 is a diagram illustrating an example of functions of the control terminal 101 to sterilize a hearing device. Referring to FIG. 6, the control terminal 101 may control various functions of the sterilizing unit 204. Such functions may be displayed through a display provided in the control terminal 101, and a user may control the functions of the sterilizing unit 204 through an input of the control terminal 101.

The control terminal 101 may control a sterilizing time, a sterilizing period, a disinfectant replacement, a sterilizing state, and a sterilizing timer setting of the hearing device. The hearing device is worn in an ear of a human and thus, there may be earwax on the hearing device. The earwax is unsanitary and may cause malfunction of the hearing device. Thus, the hearing device is sterilized to keep it sanitary.

The sterilizing time refers to an amount of time during which a disinfectant is to be sprayed on a hearing device. The sterilizing period refers to a set period to spray a disinfectant on a hearing device. The disinfectant replacement refers to an operation of replacing a disinfectant or refilling the sterilizing unit 204. The operation of refilling the disinfectant may be carried out after an amount of a disinfectant to be used to sterilize a hearing device is verified. The sterilizing state may indicate a sterilization level of a hearing device, a progress of sterilization, or whether the sterilization is completed. The sterilizing timer setting refers to a timer setting for the sterilizing unit 204 to initiate sterilizing a hearing device at a predetermined point in time.

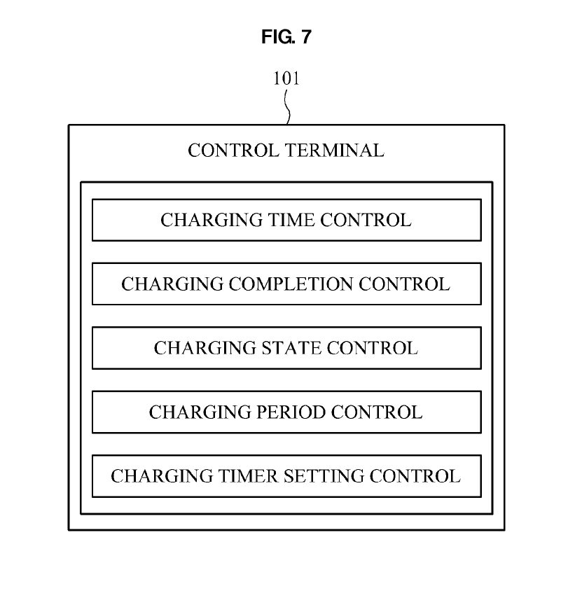

FIG. 7 is a diagram illustrating an example of functions of the control terminal 101 to charge a hearing device. Referring to FIG. 7, the control terminal 101 may control various functions of the charging unit 203. Such functions may be displayed through a display provided in the control terminal 101, and a user may control the functions of the charging unit 203 through an input of the control terminal 101.

The control terminal 101 may control a charging time, a charging completion, a charging state, a charging period, and a charging timer setting of the hearing device. A hearing device may include an internal battery, which is used to power an internal processor to transfer an external sound to an ear of a user. The internal battery of the hearing device may be charged while the hearing device is being stored in the station 102.

The battery may be charged using a variety of methods, such as, for example, charging a battery through a solar cell provided in the station 102, charging a battery through a connection to a power source provided in the station 102, or wirelessly charging a battery through a resonance between a magnet provided in the station and a coil provided in a hearing device.

The charging time refers to an amount of time during which an internal battery of a hearing device is charged. The charging completion indicates whether an internal battery of a hearing device is completely charged. The charging state indicates a power level of an internal battery of a hearing device. The charging period refers to a period set to charge a hearing device. The charging timer setting refers to a timer setting for the charging unit 203 to initiate charging a hearing device at a predetermined point in time.

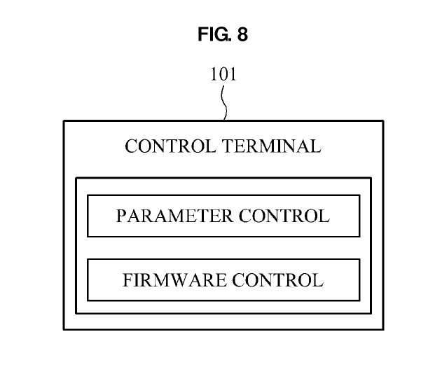

FIG. 8 is a diagram illustrating an example of functions of the control terminal 101 to set a hearing device. Referring to FIG. 8, the control terminal 101 may control various functions of the setting unit 206. Such functions may be displayed through a display provided in the control terminal 101, and a user may control the functions of the setting unit 206 through an input of the control terminal 101.

The control terminal 101 may control different functions, such as, for example, a firmware upgrade and a parameter adjustment of the setting unit 206. A hearing device may be worn in an ear of a human. Since characteristics of an ear of a human continuously change, the hearing device may need to be set to fit in an ear of a human. In addition, when software of a hearing device is upgraded, firmware may be upgraded to reflect such change.

The parameter adjustment refers to an adjustment of a frequency band that a user may hear. The firmware upgrade refers to an operation of changing a version of software of an internal processor of a hearing device to a latest version.

According to the aforementioned examples, various functions of a station configured to store a hearing device may be controlled through an external control terminal. In an example, it may be possible to store a hearing device, for example, a hearing aid, and to manage the hearing device by drying, cleaning, sterilizing, charging, and setting the hearing device.

In another example, it may also be possible to use a station by controlling operations of managing a hearing device, for example, drying, cleaning, sterilizing, charging, and setting the hearing device, through a control terminal disposed outside the station.

The processes, functions, and methods described above can be written as a computer program, a piece of code, an instruction, or some combination thereof, for independently or collectively instructing or configuring the processing device to operate as desired. Software and data may be embodied permanently or temporarily in any type of machine, component, physical or virtual equipment, computer storage medium or device that is capable of providing instructions or data to or being interpreted by the processing device. The software also may be distributed over network coupled computer systems so that the software is stored and executed in a distributed fashion. In particular, the software and data may be stored by one or more non-transitory computer readable recording mediums. The non-transitory computer readable recording medium may include any data storage device that can store data that can be thereafter read by a computer system or processing device. Examples of the non-transitory computer readable recording medium include read-only memory (ROM), random-access memory (RAM), Compact Disc Read-only Memory (CD-ROMs), magnetic tapes, USBs, floppy disks, hard disks, optical recording media (e.g., CD-ROMs, or DVDs), and PC interfaces (e.g., PCI, PCI-express, Wi-Fi, etc.). In addition, functional programs, codes, and code segments for accomplishing the example disclosed herein can be construed by programmers skilled in the art based on the flow diagrams and block diagrams of the figures and their corresponding descriptions as provided herein.

The apparatuses and units described herein may be implemented using hardware components. The hardware components may include, for example, controllers, sensors, processors, generators, drivers, and other equivalent electronic components. The hardware components may be implemented using one or more general-purpose or special purpose computers, such as, for example, a processor, a controller and an arithmetic logic unit, a digital signal processor, a microcomputer, a field programmable array, a programmable logic unit, a microprocessor or any other device capable of responding to and executing instructions in a defined manner. The hardware components may run an operating system (OS) and one or more software applications that run on the OS. The hardware components also may access, store, manipulate, process, and create data in response to execution of the software. For purpose of simplicity, the description of a processing device is used as singular; however, one skilled in the art will appreciated that a processing device may include multiple processing elements and multiple types of processing elements. For example, a hardware component may include multiple processors or a processor and a controller. In addition, different processing configurations are possible, such a parallel processors.

As a non-exhaustive illustration only, a terminal or device described herein may refer to mobile devices such as, for example, a cellular phone, a smart phone, a wearable smart device (such as, for example, a ring, a watch, a pair of glasses, glasses-type device, a bracelet, an ankle bracket, a belt, a necklace, an earring, a headband, a helmet, a device embedded in the cloths or the like), a personal computer (PC), a tablet personal computer (tablet), a phablet, a mobile internet device (MID), a personal digital assistant (PDA), an enterprise digital assistant (EDA), a digital camera, a digital video camera, a portable game console, an MP3 player, a portable/personal multimedia player (PMP), a handheld e-book, an ultra mobile personal computer (UMPC), a portable lab-top PC, a global positioning system (GPS) navigation, a personal navigation device or portable navigation device (PND), a handheld game console, an e-book, and devices such as a high definition television (HDTV), an optical disc player, a DVD player, a Blue-ray player, a setup box, robot cleaners, or any other device capable of wireless communication or network communication consistent with that disclosed.

While this disclosure includes specific examples, it will be apparent to one of ordinary skill in the art that various changes in form and details may be made in these examples without departing from the spirit and scope of the claims and their equivalents. The examples described herein are to be considered in a descriptive sense only, and not for purposes of limitation. Descriptions of features or aspects in each example are to be considered as being applicable to similar features or aspects in other examples. Suitable results may be achieved if the described techniques are performed in a different order, and/or if components in a described system, architecture, device, or circuit are combined in a different manner and/or replaced or supplemented by other components or their equivalents. Therefore, the scope of the disclosure is defined not by the detailed description, but by the claims and their equivalents, and all variations within the scope of the claims and their equivalents are to be construed as being included in the disclosure.

What is claimed is:

1. An apparatus for managing a hearing device comprising:
a drying unit configured to dry the hearing device disposed in the apparatus; and
a communication unit configured to receive a control instruction from a control terminal to adjust an operating parameter of the drying unit.

2. The apparatus of claim 1, wherein the operating parameter of the drying unit comprising at least one of a drying time, a drying strength, a drying period, a drying timer setting, or a drying state of the hearing device.

3. The apparatus of claim 1, wherein a display configured to indicate an operating state of the drying unit is disposed on the apparatus.

4. The apparatus of claim 1, wherein a lighting device configured to display different colors based on an operating state of the drying unit is disposed on the apparatus.

5. A apparatus for managing a hearing device comprising:
a cleaning unit configured to clean the hearing device disposed in the apparatus; and
a communication unit configured to receive a control instruction from a control terminal to adjust an operating parameter of the cleaning unit.

6. The apparatus of claim 5, wherein the operating parameter of the cleaning unit comprising at least one of an airflow strength, a suction strength, a rotation velocity of a brush to clean the hearing device, a cleaning state, a cleaning period, or a cleaning timer setting of the hearing device.

7. The apparatus of claim 5, wherein a display configured to indicate an operating state of the cleaning unit is disposed on the apparatus.

8. The apparatus of claim 5, wherein a lighting device configured to display different colors based on an operating state of the cleaning unit is disposed on the apparatus.

9. An apparatus for managing a hearing device comprising:
a sterilizing unit configured to sterilize the hearing device disposed in the apparatus; and
a communication unit configured to receive a control instruction from a control terminal to adjust an operating parameter of the sterilizing unit.

10. The apparatus of claim 9, wherein the operating parameter of the sterilizing unit comprising at least one of a sterilizing time, a sterilizing period, a disinfectant replacement, a sterilizing timer setting, or a sterilizing state of the hearing device.

11. The apparatus of claim 9, wherein a display configured to indicate an operating state of the sterilizing unit is disposed on the apparatus.

12. The apparatus of claim 9, wherein a lighting device configured to display different colors based on an operating state of the sterilizing unit is disposed on the apparatus.

13. An apparatus for managing a hearing device comprising:
a charging unit configured to charge a hearing device disposed in the apparatus; and
a communication unit configured to receive a control instruction from a control terminal to adjust an operating parameter of the charging unit.

14. The apparatus of claim 13, wherein the operating parameter of the charging unit comprising at least one of a charging time, a charging completion, a charging period, a charging timer setting, and a charging state of the hearing device.

15. The apparatus of claim 13, wherein a display configured to indicate an operating state of the charging unit is disposed on the apparatus.

16. The apparatus of claim 13, wherein a lighting device configured to display different colors based on an operating state of the charging unit is disposed on the apparatus.

17. An apparatus for managing a hearing device comprising:
a setting unit configured to set an application of a hearing device disposed in the apparatus; and
a communication unit configured to receive a control instruction from a control terminal to adjust an operating parameter of the setting unit.

18. The apparatus of claim 17, wherein the operating parameter of the setting unit comprising at least one of a setting of a parameter or a firmware upgrade of the hearing device.

19. The apparatus of claim 17, wherein a display configured to indicate an operating state of the setting unit is disposed on the apparatus.

20. The apparatus of claim 17, wherein a lighting device configured to display different colors based on an operating state of the setting unit is disposed on the apparatus.
